(19) United States
(12) Patent Application Publication
(10) Pub. No.: US 2026/0276234 A1
(43) Pub. Date: Sep. 17, 2026

(54) METHOD, PRODUCT, AND SYSTEM FOR REGULATING FACILITY TEMPERATURE USING REAL-TIME PROCEDURAL STATE ANALYSIS

(71) Applicant: Kristian Thomas Dacey, Indian Land, SC (US)

(72) Inventor: Kristian Thomas Dacey, Indian Land, SC (US)

(21) Appl. No.: 19/465,828

(22) Filed: Jan. 30, 2026

Related U.S. Application Data
(60) Provisional application No. 63/773,298, filed on Mar. 17, 2025.

(51) Int. Cl. F24F 11/64; G16H 40/20

(52) U.S. Cl. CPC F24F 11/64; G16H 40/20

(57) ABSTRACT

Disclosed is a computer-implemented method, a computer program product, and/or a system for automated temperature control. A stream of timestamped clinical workflow events may be received from a clinical source. A stored event definition database may be accessed to classify at least one clinical workflow milestone within the stream of timestamped clinical workflow events. A current procedural phase of an operating room may be resolved by evaluating a first clinical workflow milestone against stored procedural state model. A control data structure correlating defined procedural phases with at least one corresponding temperature control may be accessed. A first target temperature may be derived from the parameter associated with the resolved current procedural phase. A second target temperature may be derived in response to a second clinical workflow milestone. Temperature control commands encoding the target temperatures may be transmitted to a facility management controller associated with the operating room.

### TECHNICAL FIELD

The present disclosure relates to automated temperature control systems, computer-implemented methods, and/or computer program products for healthcare facilities.

### BACKGROUND

Healthcare facilities may use heating, ventilation, and air conditioning (HVAC) systems to maintain specific temperature parameters within surgical operating rooms. Clinical guidelines and/or perioperative best practices may emphasize the importance of maintaining appropriate operating room temperature at different stages of a surgical procedure to support patient normothermia, reduce the risk of surgical site infections, minimize blood loss, and/or provide optimal anesthetic performance. Failure to transition temperature at appropriate times can result in patient hypothermia, clinician discomfort, workflow disruption, and/or extended room turnover times.

One drawback of existing systems may be that standard building automation systems (BAS) may use environmental sensors such as thermostats and/or return air sensors that may operate on closed-loop feedback. These sensor-based architectures may be reactive in that they may trigger HVAC adjustments only after a temperature deviation has already occurred. Because it may lag behind commanded setpoints, this reactive approach may result in latency and/or overshoot, which may cause temperature conditions to oscillate during critical procedural transitions.

Furthermore, environmental sensors may not be able to distinguish between clinically required cooling and random environmental fluctuations. Surgical workflows may be unpredictable due to emergency cases, cancellations, and/or variable physiological responses. In addition, existing systems that may rely on Protected Health Information (PHI) may introduce regulatory compliance burdens and/or cybersecurity risks. As a result, predictive models and/or machine learning models may fail when real-world workflows deviate from historical averages, leading to invalid control assumptions, in addition to potentially introducing regulatory burdens with privacy and/or cybersecurity risks.

Another drawback of existing systems may be that they may depend on manual adjustments and/or certain hardware. Manual adjustments may rely on inconsistent human memory, leading resets that can be missed for example, where rooms may remain cooled unnecessarily after a procedure, increasing energy consumption. Proprietary hardware-based systems may require the expensive installation and/or replacement of existing HVAC components, vendor lock-in, limited adaptability, and/or lack of awareness of true procedural milestones. Certain solutions may require specific controllers that introduce complexity in installation and/or failure points that may reduce system reliability.

In light of the foregoing, improvements to these and other drawbacks of existing systems are therefore desirable.

### BRIEF DESCRIPTION OF THE DRAWINGS

The accompanying drawings, which are incorporated in and constitute a part of this specification, illustrate several embodiments directed to one of ordinary skill in the art and, together with the description, serve to explain the disclosed principles. It should be noted that, in accordance with standard practice in the industry, various features are not drawn to scale. In fact, the dimensions of the various features may be increased or reduced for clarity of discussion. In the drawings:

FIG. 1 is a block diagram illustrating the computing environment and/or system architecture for automated temperature control, according to at least one embodiment of the present disclosure.

FIG. 2 is a block diagram illustrating functional modules of the automation control instructions, according to at least one embodiment of the present disclosure.

FIG. 3 is a schematic diagram illustrating exemplary internal data processing stages of the automation controller, according to at least one embodiment of the present disclosure.

FIG. 4 is a table illustrating an exemplary event definition database mapping vendor input labels to standardized system milestones, according to at least one embodiment of the present disclosure.

FIG. 5 is a table illustrating an exemplary control data structure mapping clinical workflow milestones to target temperature setpoints, according to at least one embodiment of the present disclosure.

FIG. 6 is a table illustrating an exemplary control data structure mapping specific procedure types to temperature setpoints, according to at least one embodiment of the present disclosure.

FIG. 7 is a flowchart illustrating an example of the primary logic routine, according to at least one embodiment of the present disclosure.

FIG. 8 is a flowchart illustrating an exemplary subprocess for deriving a target temperature setpoint based on the resolved procedural phase, according to at least one embodiment of the present disclosure.

FIG. 9 is a flowchart illustrating an exemplary subprocess for normalizing and/or classifying vendor-specific event labels using a stored definition database, according to at least one embodiment of the present disclosure.

FIG. 10 is a flowchart showing an exemplary subprocess for validating a clinical event against a procedural state model and/or updating the current procedural phase, according to at least one embodiment of the present disclosure.

FIG. 11 is a flowchart showing an exemplary subprocess for exception handling, according to at least one embodiment of the present disclosure.

FIG. 12 is a time sequence diagram showing an exemplary communication flow between the clinical source, the automation controller sever, and/or the facility destination, according to at least one embodiment of the present disclosure.

Like reference numerals refer to like parts throughout the several views of the drawings. Reference numbers that are repeated across plural figures are intended to identify the same features in various implementations. Other features of the present embodiments will be apparent from the accompanying drawings and from the detailed description that follows.

### DETAILED DESCRIPTION

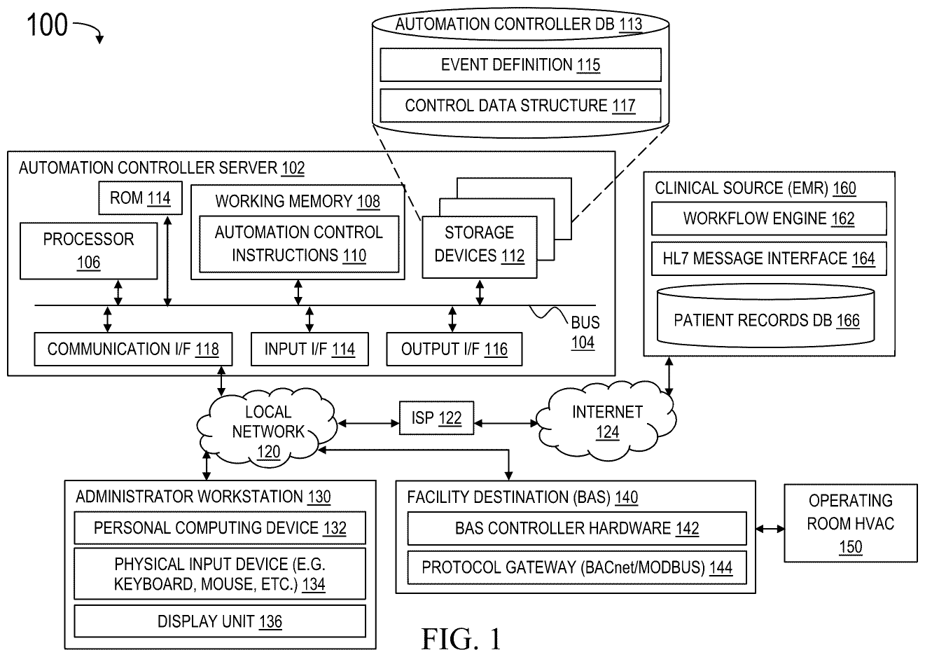

The present disclosure relates to a computer-implemented method, computer program product, and/or system for automated temperature control within a healthcare facility, such as in operating rooms. Although primarily described as middleware and with reference to surgical operating rooms, the present disclosure is not limited to such embodiments. The present disclosure may be implemented via software, hardware, middleware, firmware, and/or any combination thereof and/or may be applied to other controlled clinical environments in which procedural and/or workflow-driven temperature regulation may be desirable, such as procedure suites, imaging rooms, catheterization labs, recovery areas, etc. FIG. 1 illustrates a block diagram of an exemplary system environment 100 configured to facilitate the automated regulation of facility temperature using real-time procedural state analysis. The system environment 100 may comprise a network-enabled architecture that may function, for example, as a middleware between clinical workflow documentation and facility infrastructure management in some preferred embodiments. The system environment 100 may include an automation controller server 102, also referred to as a data processing unit. The automation controller server 102 may include a hardware processor 106 and/or a working memory 108 that may communicate via a system bus 104. The processor 106 may be a general-purpose processor, a specialized application-specific integrated circuit (ASIC), and/or another processing unit configured to execute program instructions. The working memory 108, which may comprise random access memory (RAM), may temporarily store automation control instructions 110 that, when executed by the processor 106, may cause the automation controller server 102 to perform processes and/or subprocesses.

The automation controller server 102 may be communicatively coupled to at least one storage device 112, such as non-volatile hard drives and/or solid-state drives, which may store an automation controller database 113. This database 113 may maintain data structures which may be required for the deterministic operation of the system, including, without limitation, an event definition data structure 115 used to classify workflow milestones and/or a control data structure 117 that may correlate defined procedural phases with corresponding temperature control parameters. The system may interact with external entities via a communication interface 118 connected to a local network 120. In some embodiments, the system may access remote resources via an Internet Service Provider (ISP) 122 and the Internet 124, for example, in cloud-based and/or hybrid deployment models. In cloud-based and/or hybrid embodiments, the automation controller server may reside in a remote environment and/or transmit control commands to the facility destination through encrypted tunnels to a local protocol gateway. The system may further comprise user interface components for monitoring and/or configuration. These components may include an administrator workstation 130, including a personal computing device 132, such as a desktop, laptop, tablet, mobile device, etc. These devices may allow authorized users to view system status via a display unit 136 and/or provide input via a physical input device 134, such as a keyboard, mouse, touchscreen, etc. The display unit 136 may be configured to render a dashboard displaying the current procedural phase and/or active temperature setpoint, for example.

One input to the system environment 100 may be the clinical source 160, which may be a Clinical Management System and/or Electronic Medical Record (EMR) system. The clinical source 160 may be configured to generate a stream of timestamped clinical workflow events reflecting the progression of a surgical case. Workflow events may originate from a variety of clinical integration mechanisms and/or subsystems, including, but not limited to, scheduling systems, anesthesia documentation modules, surgical case tracking systems, and/or clinical integration engines operating within the healthcare environment. In some embodiments, within the clinical source 160, a workflow engine 162 may track the status of the operating room, and/or an HL7 message interface 164, and/or an equivalent interface such as FHIR, may transmit these events to the automation controller server 102 via the input interface 114. While the clinical source 160 may contain a patient records database 166 housing protected health information (PHI), in preferred embodiments the automation controller server 102 may be configured to operate without access to PHI. At least one embodiment, which may be middleware, may ingest process filters and/or ignore patient-specific data, using only the non-identifying workflow signals to drive control logic. The automation controller server 102 may operate as a read-only consumer of workflow events and/or may not write data back to the clinical source.

The primary output of the system may be directed to a facility destination 140, such as a Facility Management Controller and/or Building Automation System (BAS). The facility destination 140 may comprise BAS controller hardware 142 which may be configured to regulate the physical environment of the healthcare facility. A protocol gateway 144 may facilitate communication between the automation controller server 102, via the output interface 116, and/or the BAS controller hardware 142. The protocol gateway 144 may translate the temperature control commands generated by the server 102 into the standard building automation protocols, such as BACnet and/or Modbus. In various embodiments, the protocol gateway 144 may communicate with a BAS and/or HVAC system using at least one of the following protocols and/or interfaces: BACnet, including BACnet/IP and BACnet MS/TP; Modbus, including Modbus TCP and/or RTU; MQTT-based interfaces supported by modern BS platforms; vendor-specific REST and/or API-based interfaces exposed by BAS vendors; and/or supervisor control interfaces provided by facility management systems.

These commands may adjust the operating room HVAC 150 equipment, controlling heating, ventilation, and/or air conditioning parameters. At least one embodiment may operate without environmental sensors. Unlike feedback loops that may rely on room temperature sensors and/or occupancy sensors, the automation controller server 102 may derive target temperatures from the stream of timestamped clinical workflow events received from the clinical source 160. As a result, at least one embodiment operating without sensors may eliminate latency and/or hardware dependency that may be seen in other systems. Unlike environmental sensors, which may only measure the result of a temperature change, workflow milestones may represent the authoritative cause and/or requirement for that change, and the system may inherently encode clinical intent.

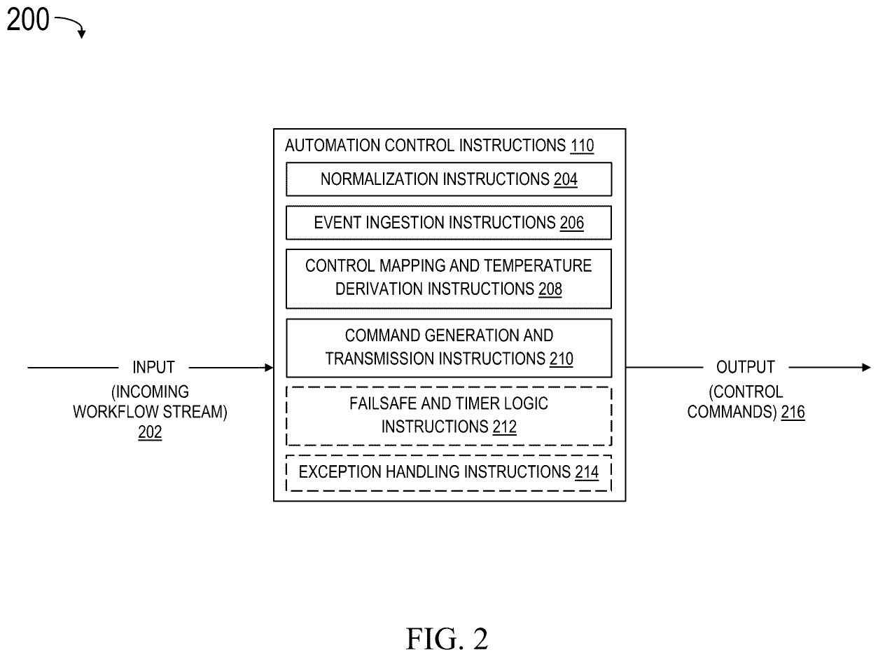

FIG. 2 is a block diagram illustrating functional modules of the automation control instructions 110, according to at least one embodiment of the present disclosure. In some embodiments, the present disclosure may be positioned logically as a middleware layer between the input 202, representing the incoming workflow stream originating from the EMR, and the output 216, representing the control commands directed to the BAS. This middleware configuration may allow the system to function as a non-invasive orchestration layer. The automation control instructions 110 residing in the working memory 108 may be organized into distinct functional modules. For example, event ingestion instructions 206 may be configured to handle the receipt of the raw input 202. These instructions may cooperate with normalization instructions 204, which may access the event definition 115 to normalize a vendor-specific event label into a canonical workflow identifier. This normalization may allow the system to interface with various EMR vendors by mapping disparate labels to a standardized internal state model.

The logic may be contained within the control mapping and/or temperature derivation instructions 208. These instructions may be configured to resolve a current procedural phase of the operating room by evaluating the normalized milestone against a stored procedural state model. Furthermore, these instructions may be configured to access the control data structure 117 to derive a first target temperature, such as a reduced temperature setpoint, and/or a second target temperature, such as a standard baseline setpoint. For example, the logic may initiate cooling upon receipt of an anesthesia readiness indicator and/or initiate warming and/or a return to baseline upon command generation and/or transmission instructions 210 may encode the value so that it may be compatible with the facility destination 140 and/or manage the transmission of the command via the output 216.

The working memory 108 may further include failsafe and/or timer logic instructions 212. These instructions may be configured to initiate a failsafe timer upon resolving a procedural phase. If a subsequent workflow milestone is not received within a defined interval, the logic may automatically force the derivation of the second target temperature to prevent the room from remaining in a cooled state indefinitely. Additionally, exception handling instructions 214 may be provided to detect anomalies, such as a communication interruption between the clinical source 160 and the facility destination 140. In the event of an interruption, these interruptions may suppress new command generation and/or maintain a last-known safe temperature setpoint. The system may not monitor, detect, nor respond to manual adjustments made at a thermostat and/or local control interface, and automation behavior may remain exclusively governed by workflow milestones and/or failsafe timing logic.

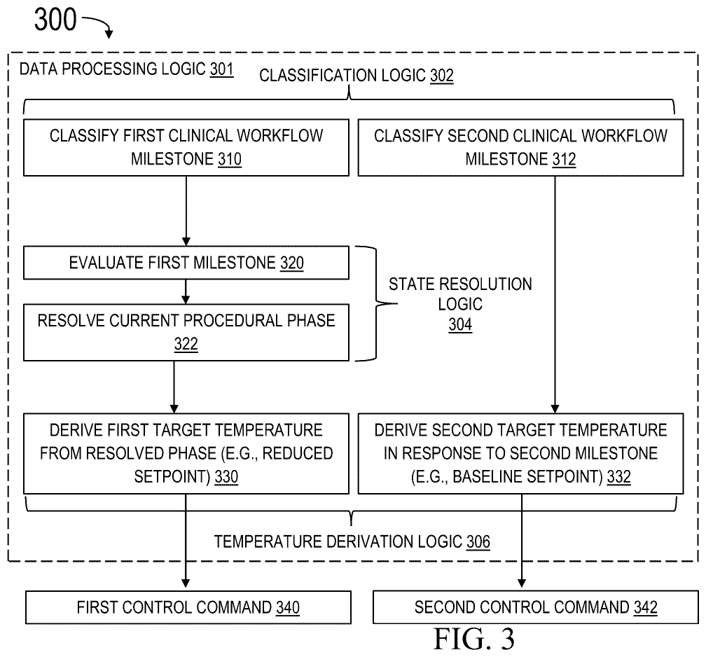

The automation controller server 102 may use specialized internal data architecture and/or memory organization configured to transform a raw stream of clinical events into deterministic control commands. As illustrated in the internal data logical processing logic 301 of FIG. 3, the process may execute a sequence of logical transformations comprising classification logic 302, state resolution logic 304, and/or temperature derivation logic 306 to bridge the gap between clinical documentation and/or building automation. The data processing logic 301 may begin with classification logic 302 configured to ingest the stream of timestamped clinical workflow events and/or perform the step 310 of classifying a first clinical workflow and/or a second clinical workflow milestone 312. This classification process may rely on a stored event definition database to normalize variable input strings into a standardized format that the system can interpret deterministically. Following classification, state resolution logic 304 may receive the standardized milestone and/or perform the step 322 of resolving a current procedural phase of the operating room by evaluating the milestones against a stored procedural state model. This logic block may be responsible for updating a logical status of an operating room from a preparatory state to a procedural state and/or validating that the sequence of events aligns with a valid surgical progression. Once the phase is resolved, temperature derivation logic 306 may access stored control mappings to identify the specific thermal regulation requirement. This logic 306 may be configured for deriving a first target temperature 330 from the control data structure in response to the resolved phase and/or subsequently deriving a second target temperature 332, such as a baseline reset, in response to a procedure completion indicator. The final output of this processing pipeline may be the generation of a first control command 340 and/or a second control command 342, which may be formatted for transmission to the facility destination.

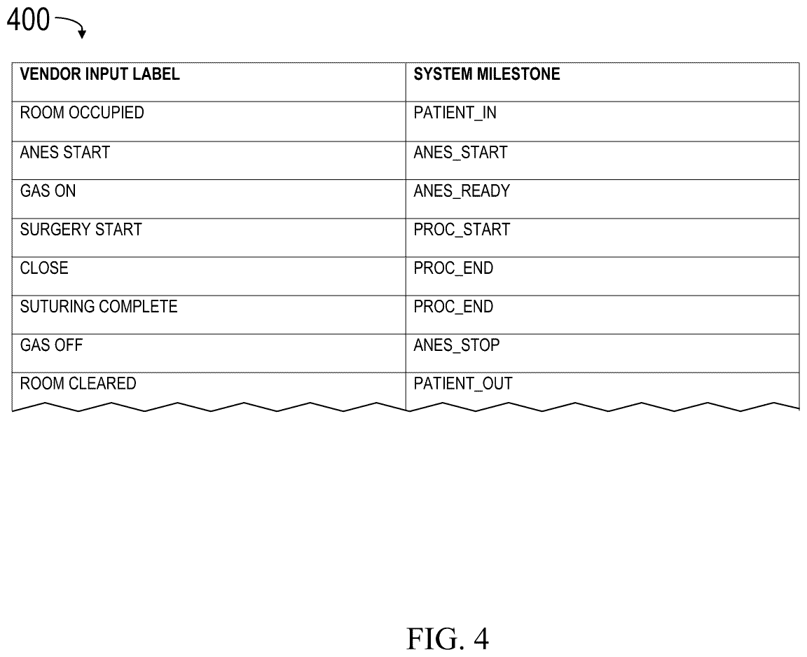

A component of this processing pipeline may be the event definition database 400 illustrated in FIG. 4, which may be stored within the non-volatile memory of the automation controller. This database 400 may allow the step of normalizing a vendor-specific event label into a canonical workflow identifier. Clinical management systems from different vendors may use disparate terminology to describe identical workflow events. To address this, the event definition database 400 may correlate a plurality of vendor input labels with corresponding system milestones. For example, the database may map a vendor-specific label such as GAS ON to the canonical anesthesia readiness indicator ANES_READY. Similarly, the database may map a label such as CLOSE or SUTURING COMPLETE to the canonical procedural completion indicator PROC_END. Other exemplary mappings stored within this data structure may include ANES START and ANESSTART, ROOM OCCUPIED and PATIENT_IN, SURGERY_START and PROC_START, ROOM CLEARED and PATIENT_OUT, etc.

For example, the PATIENT_IN milestone may indicate that the patient has entered the operating room, and upon detection, the system may be configured to set and/or maintain a warming and/or preparatory temperature suitable for patient arrival. The ANES_READY milestone may indicate that anesthesia preparation is complete and/or the operating room is ready for active anesthesia, triggering the system to proactively initiate cooling to a predefined surgical temperature setpoint so that the temperature may reach the target by the time surgical work begins.

The ANES_START milestone, indicating the initiation of anesthesia, and the PROC_START milestone, indicating the beginning of the surgical procedure, may cause the system to maintain the cooling initiated at the anesthesia readiness phase so that stable temperature conditions may be provided during active surgical work. Upon reaching the PROC_END milestone, which may indicate the completion of the surgical procedure the system may initiate warming and/or a return to a baseline and/or turnover temperature to facilitate patient emergency and/or room cleaning. The ANES_STOP milestone may indicate the termination of anesthesia care and/or may be recorded by the system for audit and/or sequencing purposes. The PATIENT_OUT milestone may indicate that the patient has exited the operating room, triggering the system to maintain the baseline temperature to support an energy-efficient turnover state.

Thus, the system may use a canonical workflow sequence which may sequentially include, without limitation: PATIENT_IN, ANES_READY, ANES_START, PROC_START, PROC_END, ANES_STOP, and/or PATIENT_OUT. This defined sequence may serve as the reference model for evaluating whether incoming events may be sequential and/or require reordering.

Thus, the system may allow the downstream control logic to operate on a consistent, standardized event sequence regardless of the specific phrasing used in the source EMR. This normalization may allow the system to function, for example, as interoperable middleware capable of ingesting diverse clinical data streams.

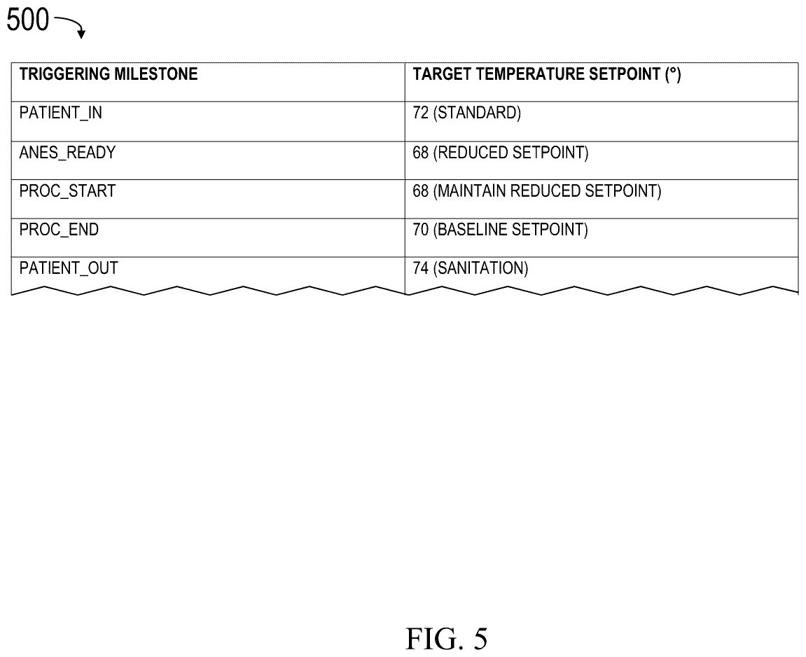

Once the incoming events are normalized, the system may rely on the control data structure 500 illustrated in FIG. 5 to determine the appropriate temperature response. This data structure 500 may correlate a plurality of defined procedural phases with at least one corresponding temperature control parameter. The system may access this data structure to enforce a workflow-driven control model that may not rely on environmental sensors. As depicted in the exemplary table of FIG. 5, the control data structure 500 may establish specific linkages between workflow milestones and temperature setpoints. A primary mapping may associate the anesthesia readiness indicator, shown as ANES_READY, with a reduced temperature setpoint, such as 68° F., for example. This mapping may be configured to cool the operating room for active surgical conditions upon the completion of anesthesia preparation. The data structure may further specify that this reduced setpoint is to be maintained through subsequent milestones, such as PROC_START. A second mapping may associate the procedure completion indicator, shown as PROC_END, with a standard baseline setpoint, such as 70° F., for example. This mapping may be configured to warm the operating room to a turnover state and/or post-procedure state upon the conclusion of the procedure. Additional exemplary mappings may associate the PATIENT_IN milestone with a standard preparatory temperature of 72° F. and/or the PATIENT_OUT milestone with a sanitation temperature of 74° F. Thus, the system allows the derivation of the first target temperature 330 and/or the second target temperature 332 to occur deterministically based on the resolved procedural phase.

In addition to the primary mapping between phase and/or setpoint, the control data structure 500 may include additional configuration columns to support complex transitions in some embodiments. These fields may include, without limitation: a Staging/Ramp Parameter, during whether the temperature change should be immediate (step-function) or gradual (interpolated over time) to prevent BAS pressure spikes; a Priority Weight, assigning a numerical value to the rule to resolve conflicts between concurrent events; and Rule Metadata, such as a specific rule identifier code. As will be appreciated, in some embodiments, every temperature control action may be strictly traceable to three coordinate data points: a specific normalized workflow event, a specific rule identifier, and/or a specific execution timestamp.

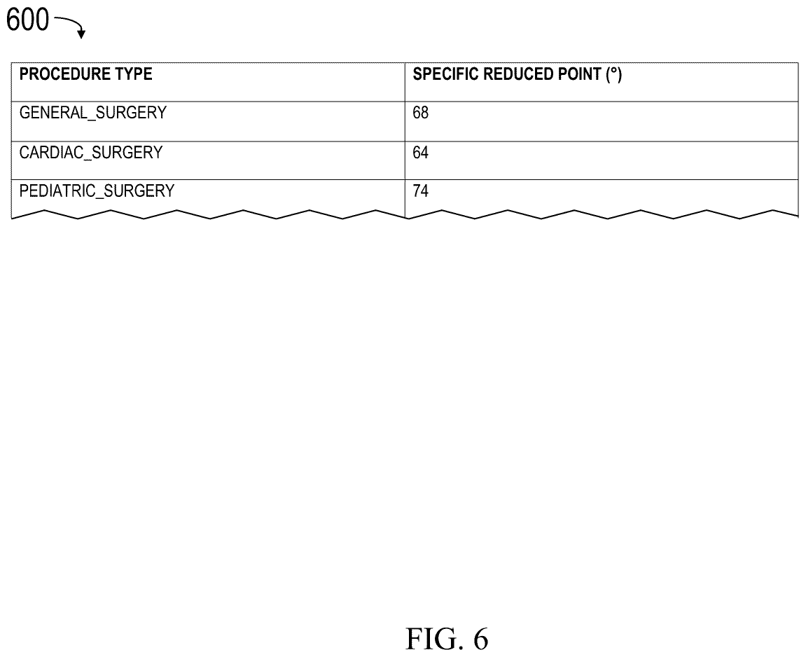

In addition to the standard control mappings, the system memory may include the procedure type mapping 600 illustrated in FIG. 6, which may allow for the customization of thermal regulation requirements based on the specific nature of the surgical case. While the general control data structure 500 may provide default settings, the stored regulation profile of FIG. 6 may allow the system to select a defined thermal regulation requirement from a plurality of profiles based on a specific procedure type identified in the workflow stream. In addition to procedure type, rule profiles may be selected and/or customized based on the specific surgical service line, the operating room designation, and/or broader institutional policies. As shown in the exemplary data structure 600, different procedure types may trigger different reduced temperature setpoints. For example, a general surgery profile may trigger a standard reduced setpoint to 68° F.; a cardiac surgery profile may trigger a lower setpoint of 64° F. to accommodate therapeutic hypothermia requirement which may be associated with cardiac procedures; and/or a pediatric surgery profile may trigger a higher setpoint, such as 74° F. to help prevent hypothermia in infants. The processor may be configured to extract the procedure type from the metadata of the incoming workflow stream and/or query this data structure to determine the precise target temperature required for the active intervention phase.

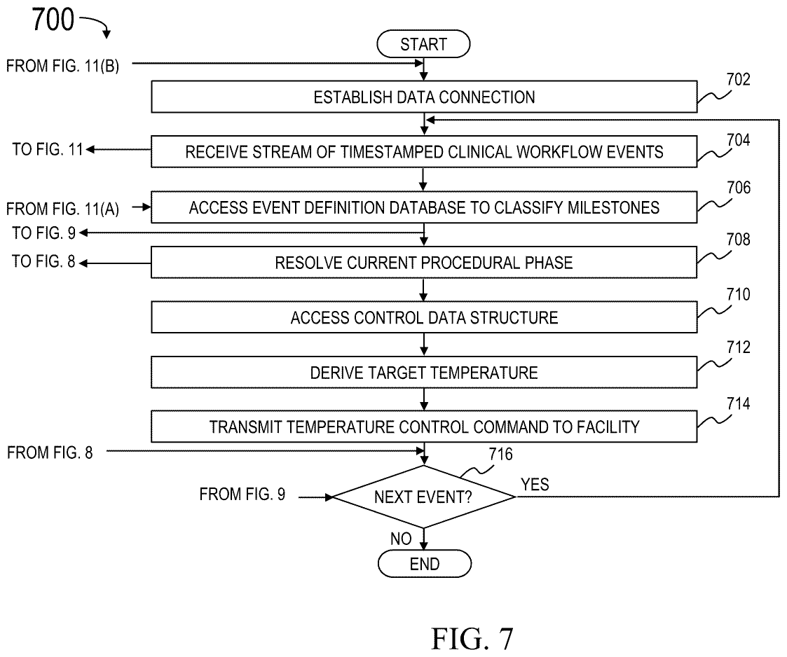

FIG. 7 illustrates an exemplary main routine logic 700 executed by the processor of the automation controller server to synchronize facility temperature parameters with clinical activity. The routine may begin at step 702 with the process of establishing a data connection between the clinical source 160 and/or the facility destination 140. This step may establish the physical and/or logical pathways required for the system to function. The processor 106 may initialize the input interface 114 to listen for incoming messages from the clinical source 160, such as an EMR and/or perioperative workflow engine. The processor 106 may also initialize the output interface 116 to establish connectivity with the facility management controller, such as BAS. Establishing this data connection may involve handshaking protocols, authentication via secure sockets, and/or initialization of a listening port for HL7 and/or FHIR message subscriptions. Thus, the automation controller server 102 may be positioned to intercept clinical signals in real-time and/or transmit control commands to the facility infrastructure.

Once the connection is established, the method may proceed to step 704, which may involve receiving a stream of timestamped clinical workflow events from the clinical management system. This step may form the primary input ingestion phase of the system. The processor 106 may continuously monitor the input stream for data packets representing discrete workflow milestones. These events may be generated by clinical staff during the normal course of perioperative documentation, for example when a nurse clicks a “Patient In Room” button and/or an anesthesiologist records a “Gas On” time. Unlike HVAC systems that may sample analog temperature readings, step 704 may involve ingesting digital, event-based data. As part of this receiving step, the system may apply filtering logic to exclude PHI, so that only non-identifying operational data, such as the event label, the timestamp, and/or the room identifier, may be processed by the downstream logic. The stream may be received asynchronously. In other words, the system may wait in a listening state until a new signal is generated by the clinical source, which may minimize computation load and/or network traffic during periods of inactivity.

Upon receipt of a raw event, the logic may advance to step 706, where the processor may perform the step of accessing a stored event definition database 400 to classify the incoming signal. Because clinical systems may vendor-specific nomenclature, the raw label received at step 704 may not be immediately intelligible to the control logic. In step 706, the processor may query the event definition database 400 to normalize the vendor-specific event label into a canonical workflow identifier. This classification process may allow interoperability because it may allow the method to function identically regardless of whether the source EMR labels an event as Patient Arrived or Occupied. This step may prepare the data for the state machine by converting variable inputs into a standardized internal vocabulary.

Following normalization, the method may proceed to step 708, which may involve resolving a current procedural phase of the operating room. In this step, the processor may evaluate the classified first clinical workflow milestone against a stored procedural state model. This logic may function as a state machine that tracks the logical status of the room, transitioning, for example, from a Preparatory State to a Procedural State or Active Intervention Phase. In a preferred embodiment, the stored procedural state model may define a set of canonical procedural states including, without limitation: Idle, Patient In, Anesthesia Ready, Anesthesia Active, Procedure Active, Procedure Complete, Patient out, and/or Anesthesia Complete. For example, the Idle state may be defined as a condition where no active surgical case is currently associated with the specific operating room. During step 708, the system may also perform validation and/or reordering operations. If an event arrives out of sequence, for example, if a Surgery Start event is received without a preceding Anesthesia Start event due to documentation lag, the logic in step 708 may update the procedural phase to reflect the most advanced state of the room. The state resolution logic may be further configured to treat a receipt of an anesthesia start indicator (ANES_START) prior to an anesthesia readiness indicator (ANES_READY) as functionally equivalent to anesthesia readiness for the purposes of initiating proactive cooling. This step may determine the true state of the operating room context, filtering out noise, duplicates, and/or ambiguous signals to establish a deterministic basis for environmental control. The outcome of step 708 may be a confirmed, validated procedural state that may control the immediate thermal requirements of the surgical environment.

Once the phase is resolved, the routine may move to step 710 and 712, which may involve determining the appropriate environmental response. At step 710, the processor may perform the step of accessing a control data structure that may correlate a plurality of defined procedural phases with at least one corresponding temperature control parameter. This lookup operation may link the abstract procedural state to a concrete physical parameter. Subsequently, at step 712, the method may involve deriving a first target temperature and/or a second target temperature based on the resolved phase.

If the resolved phase corresponds to an active intervention phase, the logic at step 712 may derive a first target temperature comprising a reduced temperature setpoint, for example, configured to cool the operating room for active surgical conditions. This derivation may result in cooling that may be initiated proactively at the onset of anesthesia. If the resolved phase corresponds to a completion phase, the logic at 712 may derive a second target temperature comprising a standard baseline setpoint configured to warm the operating room to a turnover state. This derivation may allow the system to automate the return to baseline conditions upon the conclusion of the procedure. The derivation step may also involve checking stored regulation profiles to select procedure-specific temperatures.

The control logic may also apply a deterministic rule priority process to resolve conflicts when workflow events occur in close temporal proximity. In some embodiments, the automation controller may enforce a predefined priority hierarchy among workflow milestones, such that control rules associated with procedure completion indicators may be assigned higher priority than rules associated with anesthesia readiness and/or procedure start indicators. Where multiple workflow events are received within a defined temporal window, the system may deterministically select a resulting temperature control action based on the stored priority hierarchy. As a result, the system may provide consistent outcomes for identical input conditions. The processor 106 may assign the highest priority to rules associated with the PROC_END milestone, such that a procedure completion signal may override any concurrent and/or prior cooling rules associated with ANES_READY and/or PROC_START. This priority logic may allow the conclusion of surgery to be treated as the authoritative signal for temperature reset, preventing the system from reverting to a cooling state if a delayed “Start” event is processed after a “Close” event has already occurred. Furthermore, cooling rules associated with ANES_READY may be assigned priority over preparatory warming rules, so that surgical readiness takes precedence over idle states.

After deriving the precise target temperature, the method may advance to step 714, which may involve transmitting a first temperature control command and/or a second temperature control command encoding the target temperature to the facility management controller associated with the operating room. In this step, the processor may translate the derived value into a protocol-compliant message, such as a BACnet “Write Property” request and/or a Modbus register update. The command may be sent via the output interface 116 to the BAS controller hardware 142. This transmission may alter the operation of the operating room HVAC equipment, adjusting damper positions, valve actuators, and/or fan speeds to achieve the target setpoint. Step 714 may complete the transformation of a clinical documentation event into a physical environmental change.

Finally, the routine may proceed to step 716, a decision block determining if a next event is available. If a subsequent workflow milestone is received, the logic may loop back to step 704 to process the new signal. If no new event is present, the system may wait in a monitoring state. This loop may allow the automation controller to continuously monitor the real-time procedural state of the operating room, ready to detect the next transition and/or adjust the environment accordingly.

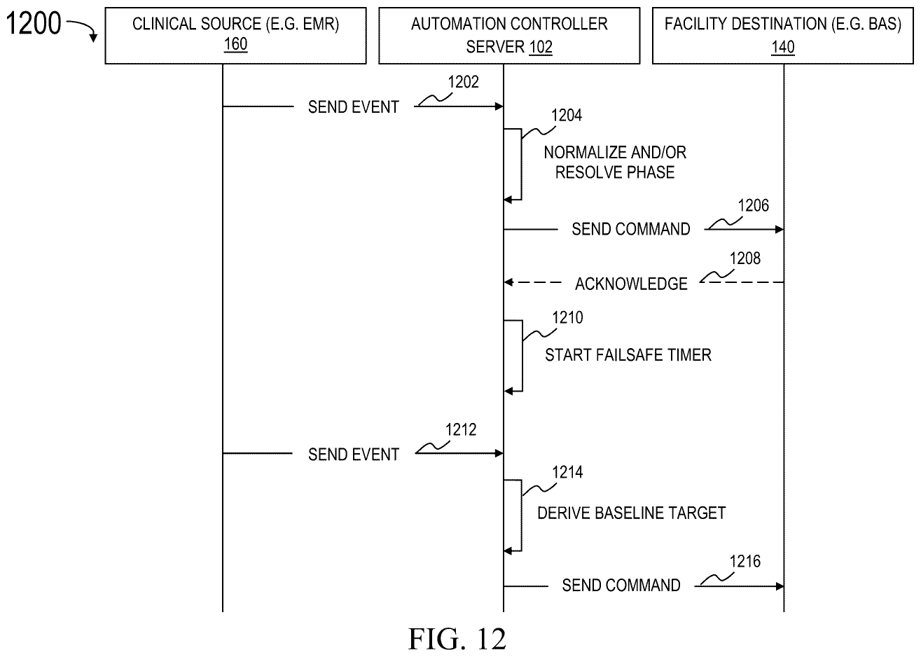

The interaction between the disparate system and/or the timing of the control logic may be further illustrated in a time sequence diagram 1200 of FIG. 12, including the communication flow between the clinical source 160 such as EMR, the automation controller server 102, and/or the facility destination 140 such as BAS over the course of a surgical procedure. The sequence may begin with the send event signal 1202. The clinical source 160 may generate a timestamped workflow event, such as an anesthesia readiness indicator (ANES_READY). This event may serve as the trigger for the automation sequence. The automation controller may receive this signal and/or perform the normalize and/or resolve action 1204. During this internal processing interval, the controller 102 may execute the classification and/or state resolution logic to determine that the room has transitioned from a setup phase to an active intervention phase. The controller 102 may identify that this specific phase transition correlates with a requirement for a reduced temperature setpoint.

Following resolution, the automation controller 102 may issue a send first command signal 1206. The signal may convey the first temperature control command, such as “Set Target 68° F.” to the facility destination 140. Thus, the system proactively initiates cooling at ANES_READY so that the room may reach the desired surgical temperature by the time the procedure formally starts. Upon receiving the command, the facility destination 140 may process the request and/or return an acknowledge signal 1208. This acknowledgement may confirm that the BAS has accepted the new setpoint and/or may adjust the HVAC equipment. The receipt of the acknowledgement signal 1208 may close the control loop for this specific transition. Verification procedures may include, without limitation: the receipt of an explicit acknowledgement message 1208 from the BAS; confirmation that the target setpoint value has been updated within the BAS controller hardware 142; and/or confirmation that a command status flag indicates successful execution. If verification fails, the system may not assume successful execution and/or instead initiate retry and/or escalation behavior. The retry logic may include, without limitation: an initial retry may be attempted after a short delay; subsequent retries may be attempted using exponential backoff; and/or after a predefined number of failed attempts, the module may report a communication failure to the failsafe and/or timer logic instructions 212.

With continued reference to FIG. 12, the automation controller may execute a start failsafe timer action 1210. This step may initiate a countdown clock in the system memory. The failsafe timer may be configured to prevent the room from remaining in a cooled state indefinitely if subsequent workflow documentation may be missing and/or delayed. This timer may run concurrently with the active intervention phase. For example, if the PROC_END milestone is not received within a defined interval following the PROC_START or ANES_START milestone, the failsafe and/or timer logic instructions 212 may infer procedure completion and/or initiate the derivation of the second target temperature based on at least one of the following exemplary deterministic conditions: the expiration of a configurable time window and/or failsafe timer measured from the last validated state; the receipt of later workflow events in the standardized sequence, such as PATIENT_OUT or ANES_STOP; and/or the receipt of administrative case closure signals from the clinical source 160.

As the procedure concludes, the clinical source 160 may generate a subsequent send event signal 1212, corresponding to a procedure completion indicator (PROC_END). This event may signal the definitive end of the surgical case. Upon receipt, the automation controller may perform the derive baseline target action 1214.

The system may recognize the PROC_END milestone as the trigger to update the logical status of the room to a post-procedure and/or turnover state. Consequently, the system may retrieve the standard baseline setpoint from the control data structure. The controller may then issue a send second command signal 1216, transmitting the second temperature control command to the facility destination 140. This command may instruct the BAS to warm the room, facilitating patient emergency and/or staff clearing activities. This inferred completion event may allow the second target temperature to be derived even in the absence of explicit documentation, so that the HVAC system returns to an energy-efficient baseline state.

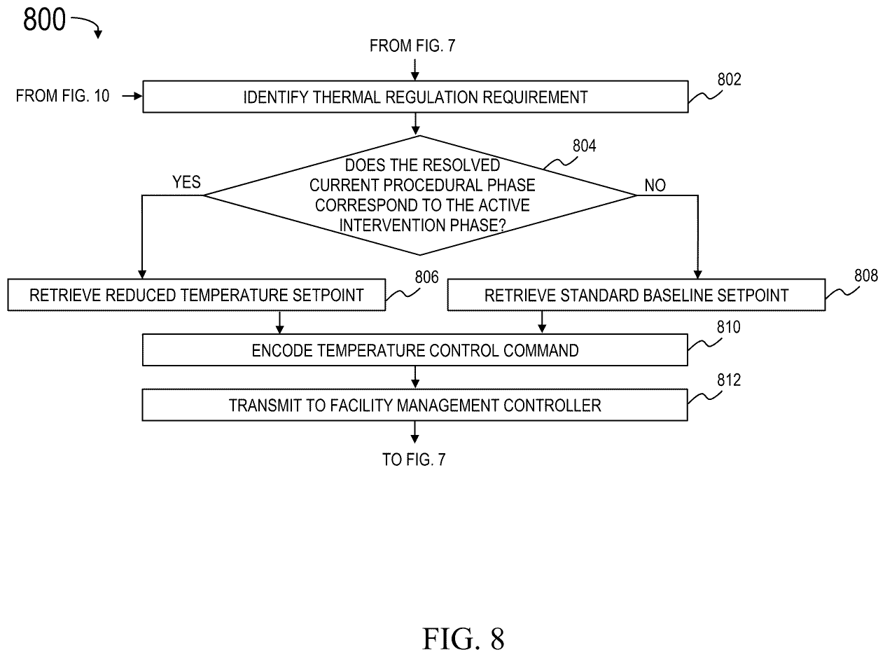

The processor 106 may be configured to execute specific logic sub-processes. FIGS. 8-10 illustrate algorithmic sub-processes for temperature derivation, event normalization, and/or state resolution, respectively. These sub-processes may provide the specific structural logic required to transform raw, potentially ambiguous clinical data into precise environmental control commands.

FIG. 8 illustrates an exemplary temperature derivation logic 800 (or temperature derivation instructions) executed by the processor 106 to determine the precise thermal regulation requirement associated with a resolved procedural phase. The subprocess may begin at step 802, where the processor 106 may perform the step of identifying a thermal regulation requirement based on the current procedural phase resolved by the state machine. In this step, the processor 106 may query the control data structure 117 to determine if the current status of the operating room controls a derivation from the building's default ambient settings. For example, this identification process may involve reading the metadata associated with the resolved phase and/or check for associated temperature flags.

Next, at step 804, the logic may enter a decision block to determine the nature of the required regulation. The processor may determine whether the resolved current procedural phase may correspond to the active intervention phase. For example, this step may involve comparing the current state ID against a list of defined surgical states.

If the determination at step 804 is affirmative (“YES”), indicating that the room may be in a state such as Anesthesia Ready, the logic may proceed to step 806. At step 806, the processor may perform the step of retrieving a reduced temperature setpoint from the control data structure. For example, the processor 106 may extract a value such as 68° F. (or a specialty-specific value like 64° F. for cardiac cases, for example) from the stored configuration profile. The logic may allow that this reduced setpoint is maintained for the duration of the active intervention phase.

If the determination at step 804 is negative (“NO”), indicating that the room has transitioned to a completion phase and/or a turnover state, the logic may proceed to step 808. At step 808, the processor may perform the step of retrieving a standard baseline setpoint from the control data structure 117. In this step, the system may identify the appropriate recovery and/or sanitation temperature required to warm the operating room to a turnover state. This branching logic may allow the system to distinguish between the need to cool the patient during surgery and the need to warm the environment for emergence and/or cleaning.

Once the specific temperature value is retrieved, the sub-process may advance to step 810, where the processor may perform the step of encoding the temperature control command. This may involve formatting the retrieved setpoint into the specific payload structure required by the facility destination. Finally, at step 812, the processor 106 may execute the transmission of the encoded command to the facility management controller, physically altering the HVAC operation.

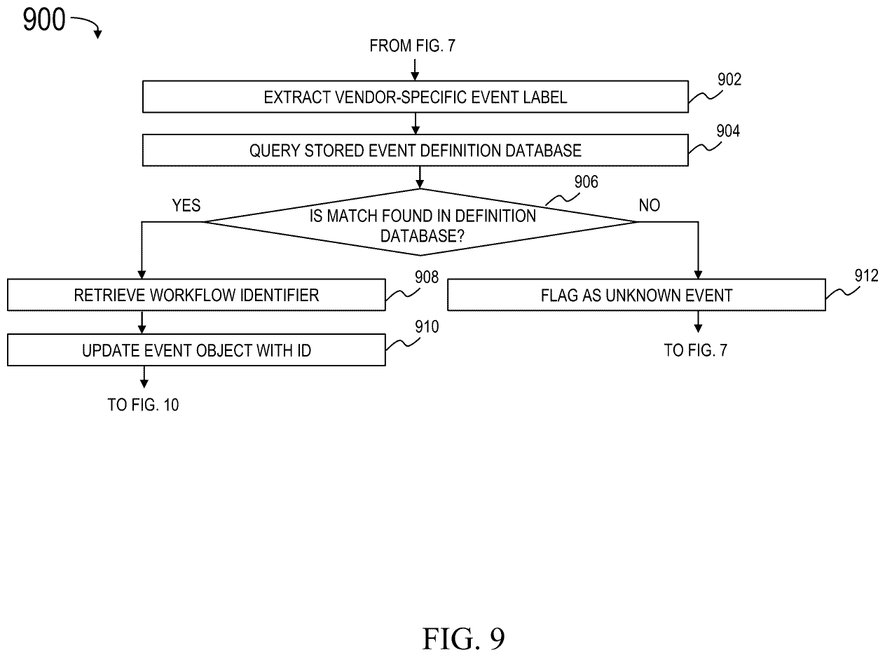

FIG. 9 illustrates an exemplary normalization logic 900 executed by the processor 106. The sub-process may begin at step 902, where the processor 106 may parse the incoming data packet to perform the step of extracting the vendor-specific event label. For example, the processor 106 may read the HL7 message segment to isolate the raw string provided by the EMR.

At step 904, the processor 106 may access the non-volatile memory to perform the step of querying the stored event definition database 400. The processor 106 may execute a lookup operation, searching the “Vendor Input Label” column of the database of FIG. 4, for example, for a string matching the extracted label.

The logic may proceed to a decision block at step 906, where the processor may determine if a match has been found in the definition database. If the event label corresponds to a known entry, the process may move to step 908. At step 908, the processor may retrieve the corresponding canonical workflow identifier (and/or system milestone) from the database. For instance, if the input label was “GAS ON,” the processor may retrieve the internal identifier ANES_READY. If the input label was “CLOSE,” the processor may retrieve PROC_END, as shown in FIG. 4. This step may map diverse input to the standardized event sequence. Then, at step 910, the processor 106 may update the event object with the retrieved ID, transforming the raw, vendor-specific signal into a standardized system command that downstream logic can interpret deterministically.

If, however, the determination at step 906 is negative (“NO”), meaning the EMR has sent a label not recognized by the system, the logic may proceed to step 912. At step 912, the processor 106 may flag the signal as an unknown event. In this scenario, the system may log the unknown string for administrative review but may suppress any state changes and/or temperature commands to prevent unpredictable behavior.

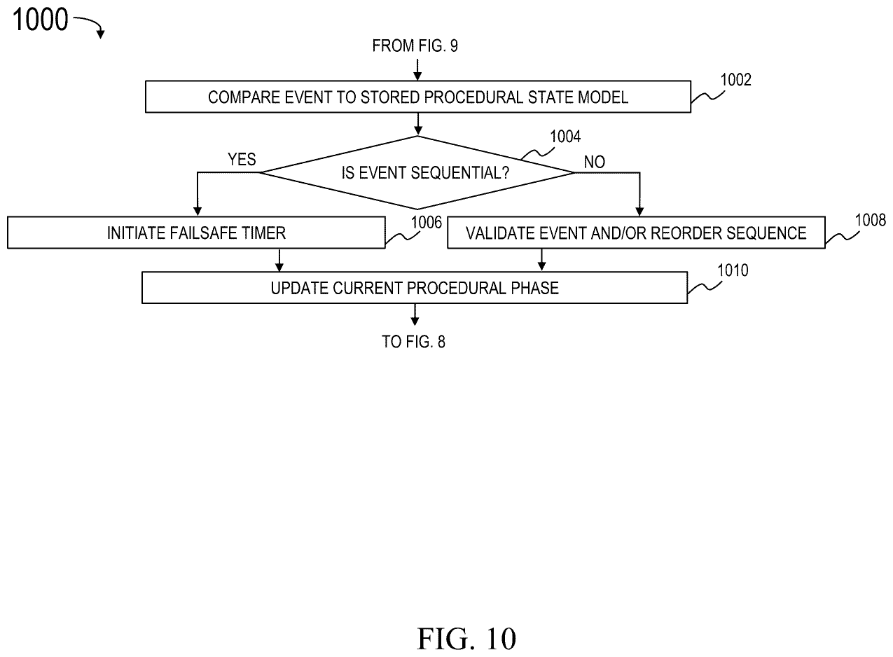

FIG. 10 illustrates an exemplary state resolution logic 1000 (or state resolution instructions). This logic may be responsible for resolving a current procedural phase and/or handling the complexities of real-world clinical documentation, including out-of-order events and/or redundancy. The sub-process may begin at step 1002, where the processor may compare the newly normalized workflow milestone to a stored procedural state model. This model may define the valid transitions between states. The processor 106 may evaluate whether the incoming event logically follows from the current state of the operating room.

The flow may move to a decision block at step 1004, where the processor 106 may determine if the event may be sequential. This check may involve verifying if the new event follows the canonical sequence.

If the event is sequential (“YES”), the logic may proceed to step 1006, where the processor may perform the step of initiating a failsafe timer. This timer may be stored in memory and/or associated with the active phase. If the next expected event is not received within a defined duration, the expiration of this timer may trigger the automatic derivation of the second target temperature (baseline reset), preventing the room from remaining in a cooled state indefinitely due to a missing completion signal. Then, the logic may proceed to step 1010, where the processor 106 may perform the step of updating the current procedural phase in memory, for example, changing the status from Idle to Active Intervention.

However, if the determination at step 1004 is negative (“NO”), indicating a deviation from the expected sequence, the logic may advance to step 1008. At step 1008, the processor 106 may execute the reordering and/or validation logic. For example, if the system receives a PROC_START event but has not yet received an ANES_READY event, for example due to documentation lag, the logic at step 1008 may analyze the timestamps and/or event hierarchy. The processor 106 may be configured to infer that if the procedure has started, anesthesia must be ready. Therefore, at step 1008, the system may reorder a plurality of events by validating the current event and/or logically filling the gap, which may advance the state to the most critical active phase. This may allow the cooling to not be withheld because a nurse may have forgotten to click a button, for example.

Furthermore, step 1008 (and/or the comparison at step 1002) may include a redundancy check. The processor 106 may be configured to compare a newly received workflow signal against the logical status of the room. If the system is already in the Active Intervention Phase and/or receive a duplicate ANES_READY signal, the logic at step 1008 may suppress the generation of a new control command. This may prevent the system from flooding the facility controller with redundant instructions, improving efficiency of network operation. Then, the system may proceed to step 1010.

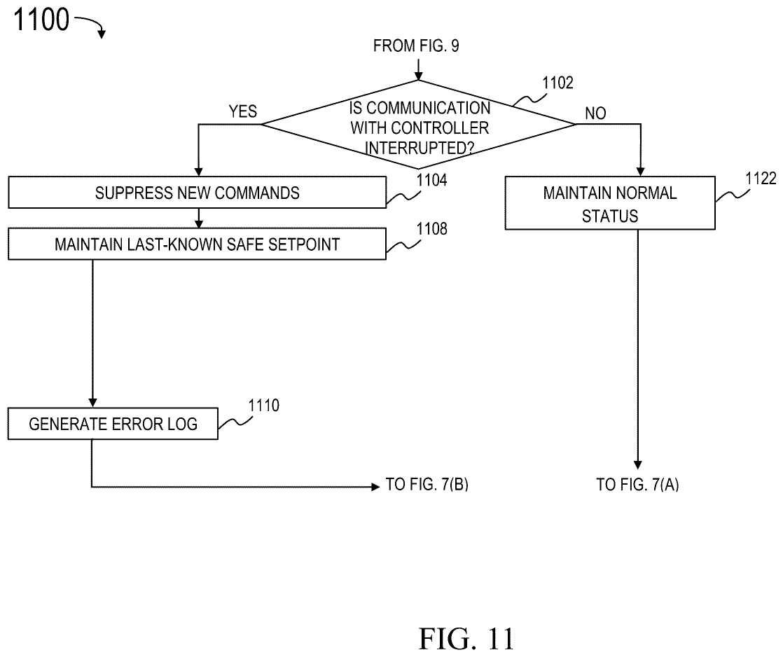

The automation controller server 102 may use an exception handling and/or failsafe logic routine. FIG. 11 illustrates an exemplary exception handling logic 1100 executed by the processor 106 to manage anomalies such as network outages, loss of signal integrity, and/or absence of expected workflow milestones. The automation controller may not evaluate thermostat state, user-entered setpoints, and/or manual control inputs, and/or may operate based on workflow milestones and/or failsafe timeout logic. The exception handling sub-process may begin at step 1102, when the processor 106 performs the step of detecting a communication interruption between the clinical source 160 and/or the facility destination 140. In this step, the processor 106 may evaluate the connectivity status of the output interface and/or the protocol gateway. This detection may involve monitoring for a heartbeat signal from the BAS, checking for TCP/IP socket disconnects, and/or identifying a failure to receive an acknowledgement signal following a previous command transmission.

If the determination at step 1102 is affirmative (“YES”), indicating that the link to the HVAC hardware may be comprised, the logic may proceed to step 1104. At step 1104, the processor 106 may execute instructions for suppressing generation of at least one new temperature control command. This suppression logic may prevent command flooding and/or the transmission of partial data packets that could cause unpredictable behavior in the facility infrastructure. The system may lock the output stream to prevent the accumulation of unverified commands in the network buffer.

At step 1108, the processor 106 may execute instructions for maintaining a last-known safe temperature setpoint stored in the memory. In some embodiments, the system may enter a degraded operating mode during such conditions, wherein automated temperature transitions may be suspended and/or constrained to predefined safe bounds until system integrity is restored. During degraded operation, the automation controller may suppress new control commands but still continue to monitor workflow signals and/or system status. Accordingly, the system may hold the operating room at the specific temperature defined by the most recent successfully validated workflow state. For example, if communication is lost during an active cardiac procedure, the system may maintain the derived reduced temperature setpoint to provide for patient safety.

The logic may further proceed to step 1110, where the processor 106 may generate an error log entry, recording the timestamp, the specific nature of the interruption, and/or the active procedural phase at the time of failure. In addition to logging, the failsafe controller may generate real-time alerts and/or notifications directed to facilities management personnel so that rapid human intervention may be provided when automated communication fails. This data may be written to the audit log database to support post-incident analysis and/or compliance reporting. Once the interruption is resolved, the system may be configured to resume normal operation.

If communication with the controller is not interrupted (“NO” at step 1102), the logic may proceed to step 1122, maintaining the Normal status and/or allowing the flow to return to the main routine, for example the exemplary process shown in FIG. 7, for standard processing.

In addition to handling explicit exceptions, the system may use a temporal failsafe mechanism to address scenarios where events may be missing. This sub-process, as illustrated at step 1006 in FIGS. 10 and 1210 in FIG. 12, may allow the operation room to not remain in a cooled state indefinitely if the clinical documentation stream may be interrupted.

Upon resolving a current procedural phase, the processor 106 may execute step 1006 (and/or step 1210) to initiate a failsafe timer or safety timer stored in the working memory. This timer may be configured with an elapsed duration threshold that may represent the maximum expected duration for the specific procedure type.

Upon generation of this inferred event, the sub-process may proceed to automatically deriving the second target temperature from the control data structure 117. This action may be configured to automatically force the logical status to a post-procedure state. Consequently, the system may command the facility controller to return the room to the standard baseline setpoint. As a result, even in the absence of closing documentation, the HVAC system may return to an energy-efficient turnover state, preventing energy waste and/or resetting the room for the next patient.

### Alternative Embodiments

Alternative configuration of the rule-based translation engine may use a staged transition profile to manage temperature changes more gradually. The system may be configured to execute a staged transition. In this embodiment, the processor 106 may retrieve a stored profile configured to gradually adjust a temperature of the operation room over a defined interval. The processor 106 may calculate a series of intermediate temperature setpoints interpolated between the current value and/or the target thermal regulation requirement. For example, if the defined interval is 20 minutes, the system may issue incremental commands to lower the setpoint by 1° F. every 5 minutes until the reduced temperature setpoint is achieved. This staged approach may be advantageous in facilities with sensitive HVAC infrastructure because it may prevent sudden spikes in energy demand and/or mitigate the risk of oscillation in the air handling units. Whether staged or immediate, these transitions may remain deterministic and/or rule-driven so that the system may calculate and/or issue a series of intermediate temperature setpoints interpolated between the current ambient value and/or the target thermal regulation requirement to optimize patient emergence comfort and/or overall energy efficiency.

In some embodiments, the system may incorporate automation control instructions 110 that may cause the processing unit to write the updated logical status to a non-volatile memory upon each transition. This operation may allow the workflow state machine to not rely only on volatile working memory (RAM). In the event of a power failure, server reboot, and/or software update, the system may be configured to perform a restoration routine immediately upon startup. This routine may involve reading the last recorded procedural phase from the non-volatile database and/or restoring the logical status to that state. By doing so, the system may maintain the continuity of a thermal regulation environment. This logic may also apply to the failsafe timer. For example, if a timer was active prior to the outage, the system may calculate the elapsed downtime and/or resume the countdown from the corrected value.

The automation controller may support scalability across large healthcare enterprises through a multi-instance configuration. The system may be configured to manage separate workflow state machines for a plurality of operating rooms concurrently. In this embodiment, the automation controller server 102 may act as a centralized host for multiple logical instances of the control logic. The input interface 114 may receive an aggregated stream of workflow events from the clinical management system, where each event may contain a location identifier, such as OR-01, OR-02, etc. The processor 106 may use a routine logic to direct each event to the specific state machine instance associated with that location. In embodiments supporting multiple operating rooms, the automation controller server 102 may instantiate a separate, independent instance of the workflow state machine for each respective operating room to allow concurrent automation across multiple procedures without cross-room interference. These operating rooms may be controlled concurrently and/or independently. As a result, a Procedure Completion event in the first operating room may trigger a warming command for that room's specific BAS controller; a simultaneous Anesthesia Readiness event in a second operating room may trigger a cooling command for the second room's controller. Thus, the procedural state of one surgical suite may not influence the temperature parameters of an adjacent suite.

The system may be further configured to execute multi-zone coordination for complex environments. In this embodiment, the BAS/HVAC communication module may map a single operating room identifier to a plurality of distinct BAS control points, such as a Variable Air Volume damper, a hydronic reheat coil, and/or a dedicated exhaust fan. Upon derivation of a single target temperature, the processor 106 may issue coordinated commands to all associated control points simultaneously to achieve the desired environmental state. This one-to-many mapping capability may allow the system to effectively regulate temperature in surgical suites containing disparate HVAC sub-system.

In some embodiments, the system may comprise a visualization and/or compliance subsystem to facilitate human oversight. In certain embodiments, the system may store historical operational data, including prior workflow milestone sequences, timing intervals, override occurrences, and/or temperature transition performance metrics. Such historical data may be used to refine configuration parameters, validate timing assumptions, and/or optimize staged transition profiles. However, control decisions may remain driven by real-time workflow milestones and/or stored deterministic rules. The system may include a dashboard module configured to render the real-time procedural state and/or the active temperature setpoint on a display unit. This dashboard may provide clinical and/or facilities staff with immediate visual confirmation that the automation may be synchronized with the surgical workflow. For example, the dashboard may allow a nurse to confirm that “OR-05” has correctly transitioned to the Active Intervention Phase and/or that the target temperature has been set to 68° F. Furthermore, the dashboard may include a timeline generator configured to display the chronological sequence of workflow milestone events. The dashboard may be configured to display the active temperature setpoint, the specific rule, and/or procedural milestone that triggered the selection, providing an auditable record of all temperature changes.

In some embodiments, the system may implement a Role-Base Access Control (RBAC) architecture. RBAC may be separated by functional utility. For example, the system may define at least three distinct user roles: clinical viewers, such as nurses, who may possess read-only access to the real-time dashboard to verify room status; facility operators, who mas possess diagnostic access to view BAS command logs and/or communication status; and/or system administrators, who may possess write-access to configure the Event Definition Database 115 and/or Control Data Structures 117.

In some embodiments, an audit log database may be configured to store immutable records of all system activities. This logging system may be used for post-procedure analysis and/or regulatory reporting. The system may record the explicit workflow milestone events received from the EMR and the inferred workflow milestones generated by the failsafe controller. For instance, if the system triggers a baseline reset because the PROC_END signal was missing and/or the failsafe timer expired, this inferred completion may be stamped with a unique code in the audit log. This distinction may allow the hospital administrators to differentiate between standard, documentation-driven automation and/or safety-driven failsafe interventions. An audit log may record specific metadata for every automation action. Each entry in the audit log may include, for example, the normalized canonical workflow identifier; the specific rule identifier applied by the translation engine; the specific BAS command status; and/or a distinction between explicit EMR-driven events and inferred events generated by failsafe logic.

In some embodiments, the system may be deployed in a cloud-based architecture. In such embodiment, the integration module and/or the workflow state machine may reside on remote servers connected via the Internet 124. The system may use secure, encrypted tunnels, such as VPNs, to transmit the temperature control commands from the cloud environment to a lightweight protocol gateway located within the facility's local network.

In a hybrid implementation, the system may use a distributed architecture to balance centralized management with local execution reliability. In this configuration, the workflow state machine and/or rule-based translation engine may reside in a centralized cloud environment, and a lightweight local protocol gateway may reside within the facility's local network. The centralized engine may process workflow events and/or transmit high-level control directives to the local protocol gateway, which may handle the low-latency, specific protocol negotiation with the local BAS controller.

Compared to systems that may use probabilistic modeling and/or reactive sensors, embodiments according to the present disclosure may provide technical improvements in control stability and/or security. By using a deterministic rule-based approach, the system may avoid disadvantages that may be seen in predictive algorithms, where a model may become too finely tuned to past data and/or fail to handle different scenarios. In particular, the automation controller may be configured such that identical sequences of normalized workflow inputs may produce identical control outputs, absent an authorized configuration change. This deterministic behavior may allow predictable operation, simply validation, and/or distinguish the system from adaptive and/or probabilistic control architectures that may generate variable outcomes for similar clinical scenarios. The present disclosure may create a more reliable relationship where identical workflow inputs may produce identical environmental outcomes. In addition, to address latency issues where there may be a lag between a command and a result, the embodiments of the present disclosure may treat the anesthesia readiness signal as a proactive trigger to begin cooling before the surgery starts. This preemptive timing may prevent the system from over-correcting and/or causing the temperature to swing above and/or beyond the target. Further, implementations of the present disclosure may operate without sensors, thus removing the risk of hardware degrading and/or losing accuracy over time which may cause the system to fail. Finally, by processing only workflow milestones, the system may not need to handle PHI, minimizing cybersecurity risks and/or lowering the compliance overhead.

Technical solutions described in various embodiments may be implemented through software, hardware, middleware, firmware, and/or any combination of these elements. When software is used, the implementation may take the form of a computer program product comprising one or more computer instructions and/or program codes stored on a computer-readable storage medium. These instructions are loaded and/or executed by one or more processors within an electronic device to perform the specific methods and/or operational procedures disclosed herein.

The computer-readable store medium may refer to any usable medium readable by a computer or a data storage device that integrates such media. Suitable media may include semiconductor memory devices like read-only memory (ROM), random-access memory (RAM), and solid-state drives (SSD), as well as magnetic media such as floppy disks, hard disks, and/or magnetic tapes. Additionally, optical media including compact disc read-only memory (CD-ROM) and digital video discs (DVD) may be used to store the computer program instructions required to execute the described technical solutions.

Computer instructions may be transmitted from one storage medium to another via wired connections, such as coaxial cables, optical fibers, and/or digital subscriber lines (DSL), and/or through wireless methods. For the purposes of this disclosure, the terms “programmed” and “configured” are used interchangeably to describe the setup of a processor or apparatus to execute specific logic. Components referred to as “module”, “unit”, and/or ending with an “-er” or “-or” suffix denote functional components implemented via hardware, software, middleware, firmware, and/or any combination thereof. The term “connection” encompasses both direct physical or electrical connections and indirect connections established through an intermediate medium. The execution of these programmed instructions may occur on a general-purpose computer, a dedicated computer, and/or a distributed computer network to achieve the intended functionality of the disclosed embodiments.

Ordinal terms such as “first”, “second”, “third”, etc. are used for identification and do not imply relative importance, specific quantities, and/or any particular chronological order, unless context clearly indicates otherwise. Expressions like “may be” or “may have” indicate preferred embodiments and/or are synonymous with “preferably is” or “preferably has”. The phrase “at least one” means one or more and a “plurality of” means two or more. Furthermore, any numerical word encompasses at least that quantity unless it results in a technical contradiction. Logical groupings such as “at least one of a, b, and c,” include any single element, any pair, any sub-combination, or all elements in combination. Unless the context clearly indicates otherwise, singular forms are intended to include their plural counterparts.

Although the invention has been explained in relation to its preferred embodiment, it is to be understood that many other possible modifications and variations can be made without departing from the spirit and scope of the invention. All illustrations of the drawings are for the purpose of describing selected versions of the present invention and are not intended to limit the scope of the present invention.

1. A computer-implemented method for automated temperature control within an operating room, the method comprising:
receiving a stream of timestamped clinical workflow events comprising non-identifying workflow signals, by an automation controller server from a clinical management system;
accessing a stored event definition database to classify a first clinical workflow milestone and a second clinical workflow milestone within the stream of timestamped clinical workflow events, wherein accessing the stored event definition database to classify comprises normalizing a vendor-specific event label into a canonical workflow identifier stored in a memory;
resolving a current procedural phase of the operating room by evaluating the canonical workflow identifier against a stored procedural state model, by the automation controller server executing the stored procedural state model, wherein resolving the current procedural phase comprises validating the first clinical workflow milestone despite an absence of a preceding sequential milestone in the stream of timestamped clinical workflow events and reordering a plurality of events stored in the memory to determine the current procedural phase;
accessing a control data structure that correlates a plurality of defined procedural phases with at least one corresponding temperature control parameter;
deriving a first target temperature from at least one corresponding temperature control parameter associated with the resolved current procedural phase;
deriving a second target temperature from the control data structure in response to the second clinical workflow milestone;
generating the operating room automation control commands comprising a first temperature control command encoding the first target temperature and a second temperature control command encoding the second target temperature;
transmitting the operating room automation control commands to a facility management controller associated with the operating room to adjust the operating room heating, ventilation, and air conditioning (HVAC) equipment controlling heating, ventilation, and/or air conditioning parameters to modify a temperature of the operating room based on the resolved current procedural phase for the operating room automation control; and
maintaining automated control operation of the operating room automation control based on workflow milestone events and failsafe timing logic,
wherein the first temperature control command and the second temperature control command are generated from the stream of timestamped clinical workflow events,
wherein the automated control operation is performed without using environmental-sensor feedback as a control basis, and without using feedback signals from thermostats or manual control interfaces as a control basis, and
wherein maintaining the automated control operation based on workflow milestone events and failsafe timing logic is done without detecting, monitoring, or responding to manual temperature adjustments as a control input.

2. (canceled)

3. The computer-implemented method of claim 1, wherein the first clinical workflow milestone comprises an anesthesia readiness indicator, and wherein the first target temperature comprises a reduced temperature setpoint retrieved from the control data structure and configured to cool the operating room for active surgical conditions upon the completion of anesthesia preparation.

4. The computer-implemented method of claim 1, wherein the second clinical workflow milestone comprises a procedure completion indicator, and wherein the second target temperature comprises a standard baseline setpoint retrieved from the control data structure and configured to warm the operating room to a turnover state upon the conclusion of the procedure.

5. (canceled)

6. The computer-implemented method of claim 1, further comprising: initiating a failsafe timer stored in a memory upon resolving the current procedural phase; and automatically deriving the second target temperature from the control data structure based on expiration of the failsafe timer if a subsequent clinical workflow milestone is not received.

7. The computer-implemented method of claim 1, further comprising: detecting a communication interruption between the clinical management system and the facility management controller; and suppressing generation and transmission of at least one new temperature control command while maintaining a last-known safe temperature setpoint stored in a memory until the communication interruption is resolved.

8. (canceled)

9. The computer-implemented method of claim 1, wherein deriving the first target temperature and deriving the second target temperature comprise a staged transition profile stored in a memory and configured to gradually adjust the operation of the operating room HVAC equipment, adjusting damper positions, valve actuators, and fan speeds to modify and achieve the first target temperature and the second target temperature of the operating room over a defined interval.

10. A non-transitory computer-readable storage medium for clinical facility automation control, the non-transitory computer-readable storage medium having program instructions embodied therewith, wherein the clinical facility is an operating room, the program instructions executable by a processing unit to cause the processing unit to perform a method comprising:
establishing a data connection between a clinical workflow source and a facility management destination;
processing a plurality of incoming workflow signals to identify a standardized event sequence, the standardized event sequence including at least a first indicator, wherein processing the plurality of incoming workflow signals to identify the standardized event sequence comprises normalizing vendor-specific workflow labels into canonical workflow identifiers;
updating a logical status of the operating room from a preparatory state to a procedural state in response to the first indicator by evaluating the canonical workflow identifiers against a stored procedural state model by an automation controller server executing the stored procedural state model;
accessing a stored configuration profile that associates the procedural state with a defined thermal regulation requirement, wherein the first indicator is associated with a demand for a reduced temperature setpoint;
generating a facility infrastructure command for the operating room automation control corresponding to the defined thermal regulation requirement;
outputting, to the facility management destination via a building automation protocol, a the facility infrastructure command for the operating room automation control corresponding to the defined thermal regulation requirement to adjust the operating room heating, ventilation, and air conditioning (HVAC) equipment controlling heating, ventilation, and/or air conditioning parameters to modify a temperature of the operating room; and
maintaining automated control operation of the operating room automation control based on workflow signals and failsafe timing logic,
wherein the facility infrastructure command for the operating room automation control corresponding to the defined thermal regulation requirement is generated from the plurality of incoming workflow signals,
wherein the automated control operation is performed without using environmental-sensor feedback as a control basis, and without using feedback signals from thermostats or manual control interfaces as a control basis.

11. The non-transitory computer-readable storage medium of claim 10, wherein the standardized event sequence further includes a second indicator, and wherein the program instructions further cause the processing unit to: update the logical status of the operating room from the procedural state to a post-procedure state in response to the second indicator; retrieve a standard temperature setpoint associated with the post-procedure state from the stored configuration profile; and output a second facility infrastructure command for the operating room automation control configured to drive the facility management destination to the standard temperature setpoint.

12. The non-transitory computer-readable storage medium of claim 11, wherein the program instructions further cause the processing unit to: initiate a safety timer in response to the first indicator; and force the logical status to a post-procedure state if a subsequent workflow signal is not received prior to the expiration of the safety timer.

13. (canceled)

14. The non-transitory computer-readable storage medium of claim 11, wherein the second indicator is an inferred completion event generated by the processing unit based on an elapsed duration threshold measured from the first indicator.

15. The non-transitory computer-readable storage medium of claim 13, wherein the program instructions further cause the processing unit to: write the updated logical status to a non-volatile memory upon each transition; and restore the logical status from the non-volatile memory following a system restart to maintain continuity of a thermal regulation environment.

16. The non-transitory computer-readable storage medium of claim 14, wherein the standardized event sequence further includes a third indicator received subsequent to the first indicator and prior to a procedure-end indicator, and wherein the program instructions further cause the processing unit to:
validate the third indicator as a confirmation of the procedural state; and maintain the facility infrastructure command for the operating room automation control corresponding to the demand for the reduced temperature setpoint.

17. The non-transitory computer-readable storage medium of claim 10, wherein the first indicator comprises a metadata field defining a specific procedure type, and wherein the program instructions further cause the processing unit to: select the defined thermal regulation requirement from a plurality of stored regulation profiles based on the specific procedure type, such that different procedure types trigger different reduced temperature setpoints.

18. The non-transitory computer-readable storage medium of claim 17, wherein the program instructions further cause the processing unit to: compare a newly received workflow signal against the logical status of the room; and suppress generation of the facility infrastructure command for the operating room automation control if the newly received workflow signal is redundant to the current logical status.

19. A system for automated temperature regulation in a clinical facility, wherein the clinical facility is an operating room, the system comprising:
a data processing unit having a processor and a memory, the data processing unit configured as a middleware layer between an external clinical management system and a building automation controller, the data processing unit further configured to:
receive a stream of clinical workflow events from an external clinical management system and normalize vendor-specific event labels within the stream of clinical workflow events into canonical workflow identifiers;
resolve a real-time procedural state of the operating room by evaluating the canonical workflow identifiers against a stored procedural state model, including validating out-of-sequence workflow events to determine the real-time procedural state;
access a stored control mapping that links a plurality of procedural phases to a plurality of target temperature setpoints, the plurality of procedural phases including at least a setup phase and an active intervention phase;
select a specific target temperature setpoint from the plurality of target temperature setpoints based on a detected transition in the real-time procedural state;
generate a control command for the operating room automation control configured to drive a climate control system to the specific target temperature setpoint, the control command for the operating room automation control being formatted for transmission via a building automation protocol;
transmit the control command for the operating room automation control to a building automation controller to adjust the operating room heating, ventilation, and air conditioning (HVAC) equipment controlling heating, ventilation, and/or air conditioning parameters to modify a temperature of the operating room; and
maintaining automated control operation of the operating room automation control based on workflow milestone events and failsafe timing logic,
wherein the data processing unit derives the specific target temperature setpoint from the stream of clinical workflow events,
wherein the automated control operation is performed without using environmental-sensor feedback as a control basis, and without using feedback signals from thermostats or manual control interfaces as a control basis.

20. The system of claim 19, wherein the data processing unit is further configured to: detect a subsequent transition in the real-time procedural state from the active intervention phase to a completion phase; select a standard temperature setpoint from the plurality of target temperature setpoints corresponding to the completion phase; and
transmit a second control command for the operating room automation control configured to drive the climate control system to the standard temperature setpoint to return the operating room to a turnover state.
